(19) United States
(12) Patent Application Publication
(10) Pub. No.: US 2006/0206047 A1
(43) Pub. Date: Sep. 14, 2006

(54) Bandage with splint

(76) Inventors: John K. Lampe, Saint Paul, MN (US); William G. Heegaard, Minneapolis, MN (US); Roger W. Heegaard, Minneapolis, MN (US)

Correspondence Address: SHERRILL LAW OFFICES, 4756 BANNING AVE, SUITE 212, WHITE BEAR LAKE, MN, 55110-3205, US

(21) Appl. No.: 11/374,248

(22) Filed: Mar. 13, 2006

Related U.S. Application Data
(60) Provisional application No. 60/661,319, filed on Mar. 14, 2005.

(51) Int. Cl. A61F 15/00; A61F 13/00

(52) U.S. Cl. 602/42; 602/58

(57) ABSTRACT

A bandage with (i) a supple backing layer having first and second major surfaces, and (ii) a longitudinally elongated elastic splint adhesively secured to the first major surface of the backing.

This application claims the benefit of U.S. Provisional Application No. 60/661,319, filed Mar. 14, 2005.

### FIELD OF INVENTION

The present invention generally relates to a medical device for bandaging parts of the human body. Specifically, the device is a disposable bandage secured to the human body or a part of the human body with reclosable fasteners. (The device could also be used on the appendages of non-human animals.) In accordance with at least one embodiment, the device could be used as a bandage for a digit. The device could consist of a shaped laminated sheet with an interior layer of non-adherent gauze for positioning over a wound; one or more intermediate layers of absorbent material for absorbing moisture and wound exudate; an exterior layer of loop material on the exterior side of the bandage; and a splint of a stiffer material sandwiched between two of the layers. The device could be secured to the digit using a combination of compression and friction created by at least one reclosable fastener with high sheer strength and low peel strength. This could allow readjustment for comfort or removal and reapplication of the bandage for wound maintenance. Other embodiments could be used on different body parts such as the head, an arm, a leg, or a foot.

### BACKGROUND OF THE INVENTION

Bandages serve many functions. These functions could include: protection of the wound from germs and foreign matter; protection from trauma that could cause additional pain; immobilization or restriction of motion to promote healing; compression to reduce swelling; creation of a better wound environment, for example, with more moisture or with the application of agents such as medication to aid in healing; or concealment of a wound for cosmetic purposes.

However, it can be difficult to apply bandages to and retain bandages on many parts of the body. Body movement, body shape, moisture, and heat can all affect bandage performance.

Wounds to the digits are one category of wounds that illustrate the many problems confronted when applying bandages to the body. Fingers are used frequently in daily activities and therefore are exposed to germs, moisture, and trauma; fingers have a significant range of motion; and the shape of fingers makes bandage retention difficult.

Like the fingers, the toes are used in daily activities such as walking and even standing. Confinement within socks and shoes can decrease sanitation and can create moisture levels that are too high. Socks and shoes can cause friction which makes bandage retention difficult and may increase discomfort.

Other parts of the body present these and other problems. Not surprisingly, the prior art of bandages is replete with attempts to overcome these problems.

In general bandages can be divided into three broad categories. First, some bandages are primary bandages intended for application directly on a wound. Such bandages typically have a sterile surface such as a gauze pad for contact with the wound, a barrier layer to protect the wound from contaminants, and some means of fastening to the body. These bandages also might contain agents such as medication for application to the wound.

A second group of bandages are secondary bandages intended for purposes other than wound management. These other purposes might include additional protection from trauma, compression, immobilization, or additional means of retention. A third group of bandages are ones that combine features of primary and secondary bandages.

Some examples of the prior art are the following: An early primary bandage for placement on fingertips is disclosed in U.S. Pat. No. 2,875,758 to Fuzak et al. The bandage discussed there has adhesive flaps and a sterile pad for placement over a wound. The flaps are wrapped around the fingertip in sequence and either stuck to the fingertip itself or to another one of the flaps. A bandage sold under the Band-Aid® brand by Johnson & Johnson called Water Block Plus® Finger-Care™ resembles the bandage disclosed in the Fuzak patent.

Another bandage for digits (and other extremities) is tubular gauze. Most tubular gauze is sold as a non-sterile dressing and therefore should be used in comb bination with other sterile gauzes when used for wounds. Tubular gauze is sold in a variety of diameters for different sized digits or appendages. To apply tubular gauze, most clinicians use specialized applicators that apply several layers to a finger or toe. U.S. Pat. Nos. 2,715,903 and 2,739,587 to Scholl teach tubular gauze applicators and methods for applying tubular gauze. Applicators similar to the one described in the patents to Scholl are still in use in hospitals and clinics today.

U.S. Pat. No. 3,695,258 to Castle describes a disposable external splint to protect and immobilize a body member. The splint consists of “a plurality of absorbent creped fibrous sheets each having a plurality of closely spaced generally parallel crimps giving the device more flexibility along one axis than along another. The device could have an absorbent layer on the interior side and could be stored in sterile packaging. Strippable adhesive tape could hold the bandage in place. The external splint taught in the Castle patent could thus serve as a primary and secondary bandage.

U.S. Pat. No. 5,036,838 to Sherman teaches a splint that is a secondary bandage. The splint consists of a “length of unbroken loop fabric” affixed to a layer breathable foam. The device could form a tube with open ends secured by a hook fastener.

U.S. Pat. No. 5,437,621 to Andrews et al. reveals a medical dressing that could be in the form of a glove or a bootie. The glove could have three layers including a non-adherent first layer, an absorbent second layer, and a waterproof third layer. The dressing could have an adjustable opening and closure means.

U.S. Pat. No. 6,139,514 discloses a tubular device with a closed distal end for use as a finger bandage. A pad on the closed distal end could maintain medication. Adhesive at the open end of the device could secure the bandage to the finger.

U.S. Pat. No. 6,258,051 to Shesol et al. describes a disposable wound dressing for delivery of a variety of standard sized gauze pads. The gauze pads are secured to a wrap made of a “loose weave material.” The wrap has a window frame with a transparent cover to allow viewing of the gauze.

U.S. Pat. No. 6,307,118 to Reich teaches a digit wound dressing. The device consists of a wrap made of a loose weave material with an “ear” for folding over the tip of the finger and another portion which wraps around the circumference. The wrap is secured with hook and loop fasteners. The wrap could secure a sterile gauze pad on a wound.

U.S. Pat. No. 6,399,852 to Barron discloses a bandage assembly with a sterile pad to be secured to a wound with a band member. The band member secures to the pad. The band member could be wrapped around a body part. The device revealed in the Barron patent could function as a primary and secondary bandage.

U.S. Pat. No. 6,656,142 to Lee describes a splint with an antibacterial cover, a hardening cover, and an outer cover. The device apparently could be used directly on wounds, and thus could serve as a primary and secondary bandage.

The prior art suffers from certain shortcomings or limitations. The purpose of the present invention is to overcome these and other shortcomings or limitations in the prior art.

### SUMMARY OF THE INVENTION

The present invention generally relates to a medical device for bandaging parts of the human body. Specifically, the device is a disposable bandage secured to the human body or a part of the human body with reclosable fasteners. (The device could also be used on the appendages of non-human animals.) In accordance with at least one embodiment, the device could be used as a bandage for a digit. The device could consist of a shaped laminated sheet with an interior layer of absorbent material for absorbing moisture and wound exudate; an exterior layer of loop material on the exterior side of the bandage; and a splint of a stiffer material sandwiched between two of the layers. The device could be secured to the digit using a combination of compression and friction created by at least one reclosable fastener with high sheer strength and low peel strength. This could allow readjustment for comfort or removal and reapplication of the bandage for wound maintenance. Other embodiments could be used on different body parts such as the head, an arm, a leg, or a foot.

Objects and Advantages

This invention encompasses embodiments with one or more of the following objects. The advantages and objects of this invention include those set forth below.

A bandage that is safe for use and does not cause discomfort such as irritation to the skin; that is easy and convenient to use; that can be packaged for easy storage, shipping, and opening by the user; and that is easy and economical to manufacture.

A bandage that could function as a primary or secondary bandage: intended for various parts of the body including the fingers, the hand, the wrist the arms, the neck, the head, the torso, the stomach, the legs, or the feet.

A bandage whose functions could include: protection of a wound from germs and foreign matter; protection of a part of the body from trauma; immobilization or restriction motion to promote healing; compression to reduce swelling; creation of a better wound environment, for example, with more moisture or with the application of medicaments to aid in healing; or concealment of a wound for cosmetic purposes.

A bandage that could be made of various sizes and shapes for different applications.

A bandage that could be made of one or a combination of materials including plastics (both natural or synthetic), foams, metals, cellulose based materials, woven or nonwoven fabrics, glass, or ceramic.

A bandage that could be elastic (in one or more than one direction) or inelastic.

A bandage that could be single use or limited use and could be disposed of when soiled or no longer needed.

A bandage whose shape could be formed by efficient cutting means such as ultrasonic or die cutting.

A bandage that could be efficiently manufactured using bonds, seams or joints using technologies such as ultrasonic or heat welding or adhesives.

A bandage that could use seams that do not require sewing.

A bandage that could be trimmed by the user to reduce its size without negatively affecting the performance of the bandage.

A bandage that could have fastening systems that could be altered to function with a bandage of a new size.

A bandage that could carry agents to enhance healing of a wound. Such materials might include agents that: are antimicrobial, administer medication, sanitize, clean, deodorize, relieve pain, reduce swelling, promote growth or healing of tissue, or coagulate blood.

A bandage with at least parts of the bandage that may do one or more of the following: breath, absorb liquids, or repel water.

A bandage with at least an interior layer that could be positioned over a wound.

A bandage with at least a first layer that could be sterile.

A bandage with at least a first layer that could be non-adherent to wounds.

A bandage whose entire first layer is a non-adherent surface to minimize the need to position a portion of the bandage over a wound.

A bandage with at least a second layer that could, for example, be made of a highly absorbent material to absorb moisture such as wound exudate.

A bandage with at least an outer layer that could be waterproof.

A bandage with an outer layer that could function as a target surface for a fastener such as by having loop material for hook or mushroom-type fasteners.

A bandage with a splint.

A bandage with a splint that could be connected to the bandage on a outside surface or on an inside surface, or could be embedded within a layer or layers of the bandage.

A bandage with a splint that could be removable, for example, from a sleeve.

A bandage with a splint of varying degrees of stiffness depending on the application.

A bandage with a splint whose shape changes and becomes stiffer when fastened to a finger or other body part.

A bandage with a splint that could have been created by treating the bandage material with processes such as heat or materials such as additives to stiffen the bandage material.

A bandage with a splint that, when bent into a position, could remain substantially in that bent position.

A bandage for a digit with a splint that could flex around the end of the digit.

A bandage with fasteners that could bend a splint in one direction in order to stiffen the splint in another direction.

A bandage that could be secured to a part of the body with at least one reclosable fastener where the fastener does not fasten to the skin.

A bandage that could be secured to a part of the body with at least one reclosable fastener where the fastener has low peel strength.

A bandage that could be secured to a part of the body with at least one reclosable fastener where the fastener has relatively high shear strength.

A bandage with a fastening surfaces that could be less abrasive and irritating to sensitive tissue.

A bandage with fasteners that include straps that could be elastic or inelastic.

A bandage with fasteners that include straps that could be elastic or inelastic.

A bandage with straps that could pass through loops or rings for closure of the bandage.

A bandage with fasteners intended to adjustably constrict the size of the bandage on one or both sides of a widening in a body part such as the proximal joint on a finger.

A bandage with a fastener of a size that could form an adjustable ring around the proximal phalange that could constrict the size of the bandage, especially around the sides of the finger, and prevent slippage of the bandage off the end of the finger.

A bandage that could cover all or some portion of a digit including the tip of the finger.

A bandage for a digit with sufficient three-dimensional structure to maintain itself as a tube (without the digit inserted) with an opening along a side through which the digit could be inserted.

A bandage for a digit that could be made entirely of nonwoven or combinations of woven and nonwoven materials.

A bandage for a digit that could be made without traditionally sewn seams.

A bandage that could include one or more of the following: a splint, a fastening system (including mechanical or chemical), and a nonadherent surface for covering a part of the body.

A bandage that could encircle and be secured to an appendage such as a finger with substantially all portions of the bandage contacting the body being nonadherent surfaces.

A bandage with a splint and a nonadherent surface for covering a part of the body.

A bandage for a digit with a nonadherent surface for covering a part of the body and a strap for attaching to another part of the body such as the wrist or ankle.

A bandage that could comprise one or more of the following layers: (a) a body-contacting layer comprising a first web of material; (b) an absorbent layer comprising a second web of material; (c) an outer layer comprising a third web of material; and (d) a splint affixed to at least one of the layers.

A bandage with or without a splint that a user could trim to change the size or shape of the bandage or to modify or to eliminate a fastener.

A bandage with or without a splint with markings such as perforations, printed matter, or other indicia to guide or ease the trimming of the bandage.

A bandage with our without a splint with a means for sealing the bandage around the base of the finger.

A bandage such as those described herein that could be efficiently manufactured using mechanical, chemical, or thermal bonds.

A bandage that could be assembled using various manufacturing techniques ranging from hand assembly to web converting.

A bandage such as those described herein that could be assembled or assembled and packaged in one press operation using a manufacturing process such as web converting.

A splint that could be manufactured from a material selected from the group consisting of metal, plastic, foam, ceramic, cellulose-based products, and fabric.

A splint that could be fabricated from materials in the form of wire, filaments, sheets, or rolls.

Devices contemplated by this invention could be suitable for many purposes. Only some of those uses are discussed in this specification. Many other uses are contemplated within the scope of this invention.

The above summary of the present invention is not intended to describe each illustrated embodiment, object, advantage, or use of the present invention. The figures and the detailed description that follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1A is a top view of the interior side of a bandage according to a first embodiment.

FIG. 1B is a top view of the exterior side of a bandage according to a first embodiment.

FIG. 1C is a side view (viewing parallel to the T centerline) of a bandage according to a first embodiment.

FIG. 1D is a side view of a finger with a bandage in a first “partially applied” position according to a first embodiment.

FIG. 1E is a side view of a finger with a bandage in a second “partially applied” position according to a first embodiment.

FIG. 1F is a side view of a finger with a bandage affixed to it according to a first embodiment.

FIG. 1G is a side view of a smaller finger with a bandage affixed to it according to a first embodiment.

FIG. 2A is a side view of a finger with a bandage affixed to it according to a second embodiment.

FIG. 3A is a top view of the exterior side of a bandage according to a third embodiment.

### DETAILED DESCRIPTION OF THE INVENTION INCLUDING A BEST MODE Nomenclature

101 bandage
101a index finger
101b smaller finger
102 peripheral edge of bandage
103 adhesive
103a adhesive for splint
106 interior side
107 exterior side
108 fingertip or distal end of finger
110a first fastening tab
110b second fastening tab
110c third fastening tab
110d fourth fastening tab
111 release liner
112 bending of bandage
113 bandage in first partially applied position
114 bandage in second partially applied position
118 interior layer
119 intermediate layer
120 exterior layer
121 splint
122 peripheral edge of splint
200 bandage
201a index finger
207 exterior side
208 fingertip or distal end of finger
210a first fastening tab
210c third fastening tab
220 exterior layer
221 splint
300 bandage
302 peripheral edge of bandage
307 exterior side
310a first fastening tab
310b second fastening tab
310c third fastening tab
310d fourth fastening tab
318 interior layer
320 exterior layer
321 splint
322 peripheral edge of splint
   Construction

As disclosed the invention concerns a bandage 100 and in particular a bandage 100 with a splint 121 or other stiffening member. Such a bandage 100 can be used for bandaging parts of the human body to help heal and protect wounds. However, the invention is not limited to such uses. The structure of the bandage 100 may be useful for other purposes. Other purposes might include use as a brace for therapeutic purposes, as protective pad for use in sports, or any other use where a reinforced bandage or pad might be usable and beneficial.

### First Embodiment

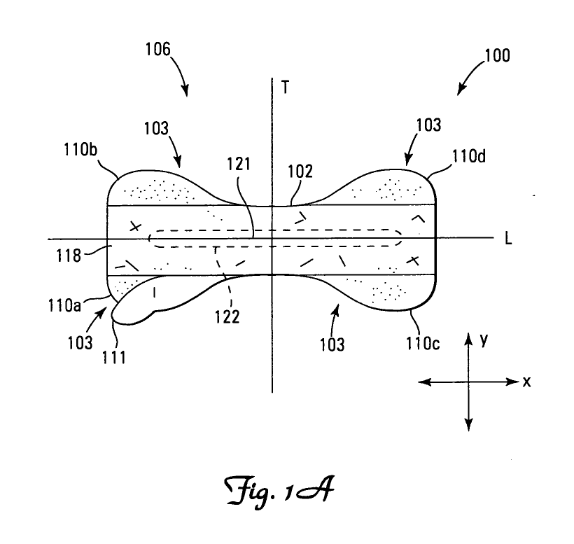

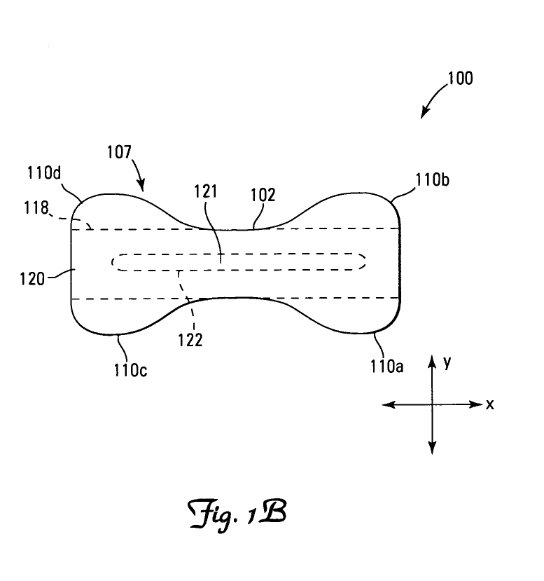

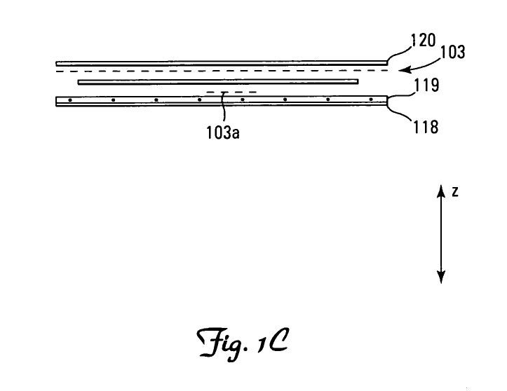

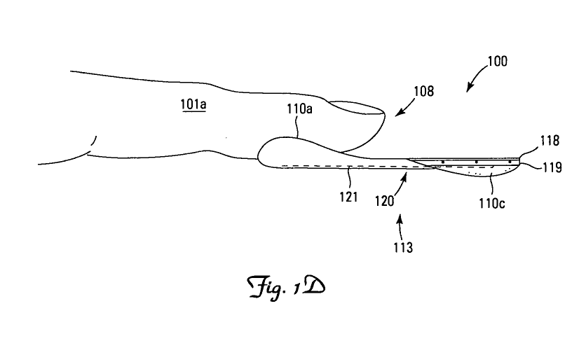

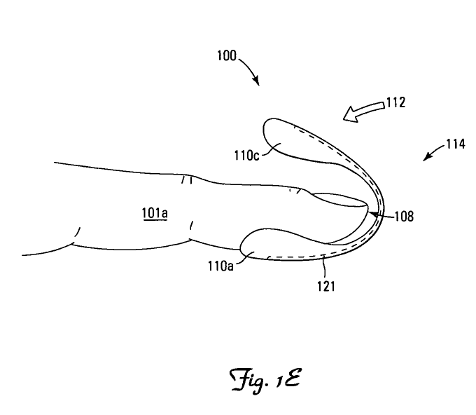

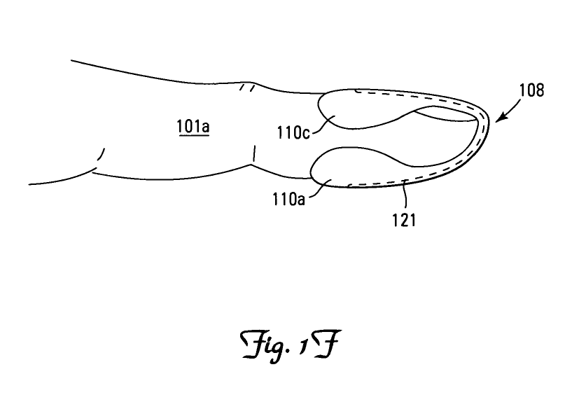

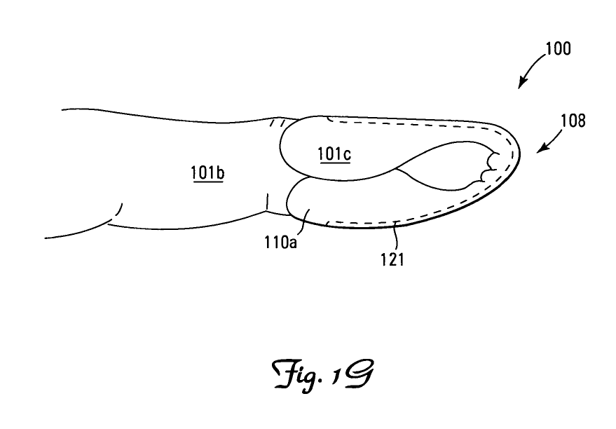

The bandage 100 shown in FIGS. 1A to 1G can be oriented with a longitudinal centerline L generally bisecting the bandage 100. The term “longitudinal” refers to a line, axis, or direction in the plane of the bandage 100 that is aligned with an “x” axis. The bandage 100 shown in FIG. 1A can further be oriented with a transverse (or lateral) centerline T that refers to a line, axis, or direction aligned with a “y” axis that is perpendicular to the longitudinal “x” axis. The bandage 100 can further be oriented with an axis in the “z” direction, which is perpendicular to the x-y plane (defined by the longitudinal and transverse centerlines), and generally corresponds to the direction associated with the thickness dimension of the bandage 100. The shape of the bandage 100 is defined by its peripheral edge 102.

The length of the bandage 100 is the maximum dimension measured parallel to the longitudinal centerline L in the longitudinal direction. The width of the bandage 100 is the maximum dimension measured parallel to the transverse centerline T in the transverse direction. The thickness of the bandage 100 is the maximum “z” direction dimension measured parallel to the “z” axis.

FIGS. 1A to IG show a bandage 100 according to the first embodiment of the invention. The bandage 100 as shown is with layers, 118, 119, 120 that can be affixed to each other preferably during manufacture.

The interior side 106 of the interior layer 118 can face the body and a wound on the body. The interior layer 118 can be made of many different materials. For most applications the interior layer 118 can preferably have properties such as being non-adherent, non-absorbent, air and liquid permeable. Such a bandage 100 can prevent the bandage 100 from sticking to a wound, can allow wound exudate to pass through to the interior layer 118, and can regulate the level of moisture present at or near a wound site. One suitable material for the interior layer 118 can be Delnet® Apertured Film from Delstar Technologies, Inc., of Middleton, Del. As discussed below, other materials could also be suitable.

For most applications the intermediate layer 119 can provide the bandage 100 with capacity to retain wound exudate migrating from the wound site. In addition, the intermediate layer 119 can provide additional structure and padding to the bandage 100.

The intermediate layer 119 can be made of many different materials. For most applications an absorbent material can be preferable. The intermediate layer 119 can thereby absorb moisture and wound exudate passing through the interior layer 118. For many applications nonwoven blends of rayon and polyester can be preferable for the intermediate layer 119 because of cost and manufacturing ease. As discussed below, other materials could also be suitable.

For many applications it may be preferable to have the interior layer 118 and the intermediate layer 119 bonded before final assembly. Such bonding is often performed by a vendor such as Delstar Technologies, Inc.

For most light duty applications a bandage 100 with an intermediate layer 119 of a thickness in the range of 0.015 to 0.070 could be suitable. As discussed below, other thicknesses could also be suitable for this or other applications.

The exterior layer 120 can be made of many materials. For most applications an exterior layer with some stretch can be preferable. A suitable material can be a tape with a polyurethane backing and an acrylate adhesive such as elastic nonwoven tape, Part No. 9907, from 3M®, Inc., of St. Paul, Minn. As discussed below, other materials could also be suitable.

A splint 121 can be attached to the bandage 100 in different ways. For example, the splint 121 can be sandwiched between the exterior layer 120 and the intermediate layer 119. Adhesive 103a can attach the splint 121 to the intermediate layer. In the preferred embodiment the splint 121 can be made of a relatively soft, malleable material such as dead soft aluminum. A splint 121 made of material such as dead soft aluminum can have minimal spring such that when the bandage 100 is bent (such as by hand) into a new position or new shape, the splint 121 can help maintain the bandage 100 substantially in that new position or shape without migrating to a previous position. The splint 121 can be pre-cut and formed into the shape shown in FIGS. 1A to 1C by a metal fabricator. Alternatively, as discussed below, the splint 121 can be cut during final assembly. For many applications such as bandaging a finger 101a, the thickness of the splint 121 can be in the range of 0.003 inch to 0.025 inch. As discussed below, other thicknesses could also be suitable.

Adhesive 103 can bond one or more layers. In many instances, adhesive can by applied to a layer such as the exterior layer 120 by a vendor before final assembly. For example, an acrylate adhesive can be applied by 3M® to its elastic nonwoven tape No. 9907. Such an elastic nonwoven tape can be used as the exterior layer 120. The adhesive 103 can then be exposed in final assembly to bond the exterior layer 120 to the intermediate layer 119 (with the splint 121 sandwiched in between). In other instances certain layers can be bonded together by a vendor before final assembly. For example, a vendor such as Delstar Technologies can bond a non-adherent interior layer 118 with an absorbent intermediate layer 119 as shown in FIG. 1C.

As shown in FIGS. 1A to 1G, fastening tabs 110a to 110d can be formed as lateral extensions of the exterior layer 120 beyond the edge of the intermediate layer 119 and the interior layer 118. On the interior side 106 of the bandage 100, as shown in FIG. 1A, the fastening tabs 110a to 110d can have adhesive 103 or another fastening material. As is common in the art, the same adhesive 103 that bonds one of the layers of the bandage 100, in this instance the exterior layer 120, can also serve as the adhesive 103 on the fastening tabs 110a to 110d. The adhesive 103 on the fastening tabs 110a to 110d can be protected before use by a release liner 111. FIG. 1A shows the release liner 111 partially removed from a fastening tab 110a. (The release liner 111 could also have two halves (not shown) that overlap the entire interior side 106 of the bandage 100 as is common in the art.).

The overall size and shape of the bandage 100 can vary depending on the application. For example, a bandage 100 for covering the area around the fingertip 108 of a typical adult can have an outside dimension in the range of 1.0 inch to 2.0 inches wide and with a length in the range of 2.0 inches to 4.0 inches. It can have a thickness of approximately 0.010 inch to 0.200 inch (although it could be thicker depending on the application). The shape of the bandage 100 can also vary depending on the application. The bandage 100 shown in FIGS. 1A to 1G can be preferable for many applications such as a bandage 100 for a fingertip 108. However, other embodiments could include the various other shapes well known in the art for fingertips and for other parts of the body.

The peripheral edge 122 of the splint 121 can lie within the peripheral edge 102 of the bandage 100. This can minimize the potential of exposure of an edge of the splint 121 during use of the bandage 100. This can also allow more of the exterior layer 120 to bond directly with the intermediate layer 119. For use with a bandage 100 for a fingertip 108, the splint 121 can have a width of approximately 0.10 inch to 1.9 inch and a length of approximately 1.4 inch to 3.65 inches. The splint could be formed into many shapes including the one shown in FIGS. 1A to 1G.

For many applications, it can be preferable to have the final assembly of the bandage 100 completed using a web converting process. Those layers not already bonded to each other in pre-assembly can be bonded to each other in final assembly. Adhesives are a common means of bonding layers of bandages 100 in a web converting process. The cutting of the bandage 100 can be done using a rotary die cutter.

If the splint 121 is cut and formed in a pre-assembly phase, the splint 121 can be placed during the web converting process using, for example, a pick-and-place machine. If the splint 121 is not cut during pre-assembly, it can, depending on its thickness, also be cut during final assembly.

FIGS. 1D, 1E, and 1F show how the bandage 100 can be applied to the index finger 101a. First the release liner 111 can be removed from the tabs 110a to 110d and the tabs 110a and 110b can be applied to the finger 101a as shown in FIG. 1D. At this stage of application, the bandage 100 can remain relatively straight in a longitudinal direction because of the splint 121. This can be considered the first “partially applied” position 113 as shown in FIG. 1D. Next, the bandage 100 can be bent 112 around the distal end 108 of the finger 101a as shown in FIG. 1D. The bandage 100 can remain in this second “partially applied” position 114 because of the bendable material in the splint 121. Finally, tabs 110c and 110d can be affixed to the finger 101a to fully apply the bandage 100.

FIG. 1F shows the bandage 100 applied to a smaller finger 101b. Tabs 110c and 110d overlap tabs 110a and 110b because of the smaller sized finger 101b.

The bandage 100 as disclosed above can offer several advantages including the following. First, the splint 121 can provide additional protection to a portion of the body that a typical soft bandage could not. This can be especially important for injured fingers which can be very sensitive to contact. Second, the splint 121 can be used to help immobilize a joint or other part of the body in a certain position. For example, the splint 121 could help maintain a joint in a bent position for therapeutic purposes.

Third, the added structure provided by the splint 121 can ease application. In many instances, adhesive tabs on bandages in the prior art fold together and become stuck or are stuck unintentionally to other surfaces. Especially for clinicians with latex gloves, this can be a problem. Part of this is due to the lack of structural support in bandages presently on the market. The splint 121 can provide structural support to the bandage 100 and make application of the tabs 110a to 110d easier. Fourth, the lack of spring in the splint 121 can help facilitate application of the bandage 100. This can allow the bandage 100 to stay without external forces in the “partially applied” positions, 113 and 114, as shown in FIGS. 1D and 1E. This can make the positioning of the tabs 110a to 110d for final application much easier. Finally, the manufacturing process for a bandage 100 with such a splint 121 can produce consistent and high quality bandages 100 in a cost-effective and efficient manner. All or large portions of the construction of the bandage 100 could be done on press using a web converting process thereby saving costs.

The above discussion discloses certain materials, construction techniques, and methods of application. However, in other embodiments, other materials, construction techniques, or methods could be used. For example, different materials can be used from those discussed above for the layers. Other non adherent materials such as Mylar® or other film materials or coatings can be used for the interior layer 118. Other materials could be used for the intermediate layer 119, including natural fibers such as cotton or woven materials or absorbent foams. The exterior layer 120 can be made of a variety of materials including polyurethane films; polyolefin films; polyvinylchloride films; ethylene vinyl acetate films; woven fabrics; or nonwoven fabrics. The splint 121 could be made of different materials such as various plastics such as polyester or foams such as EVA foams; the splint 121 could be molded or formed in other ways. Such a bandage 100 can still be within the scope of this invention.

Alternatively, in other embodiments additional layers could be added or certain layers could be eliminated. For example, the interior layer 118 can be removed completely in some embodiments. For some clinicians this might be preferable because the clinician may want to apply a separate dressing such as one with petroleum based gels. Alternatively, the bandage 100 could comprise just the exterior layer 120 with a splint 121 or just the interior layer 118, the exterior layer 120, and the splint 121. Bandages 100 such as these could be suitable as wraps that might not need separate dressing materials. Finally, any one of the layers 118 to 120 could have various additives incorporated into it. These additives could aid in healing in various ways. Such bandages 100 can still fall within the scope of this invention.

Other means of bonding the layers together can be used in other embodiments. These could range from permanent bonds between layers to bonds releasable and reclosable by hand. Moreover, the bonds could range from mechanical ones such as hook and loop, to chemical ones such as adhesive, to thermal ones such as with heat sealing materials with melt-able fibers. Such bandages 100 can still fall within the scope of this invention.

Moreover, other embodiments can encompass bandages 100 of different sizes and thicknesses with components of different sizes and thicknesses than those described above. For example, a bandage for use on the full finger might be eight inches long. A bandage for use on an adult toe might be wider, perhaps over 2 inches wide. Moreover, a bandage 100 for use as a protective device such as on an arm, a leg, etc., could have much different dimensions and a be much thicker. Such bandages can still fall within the scope of this invention

Other embodiments could include bandages with other means of fastening bandages to the body. Other kinds of adhesives can be used, including ones that release more readily or hold more firmly. Alternatively, other fastening means could be used. For example, mechanical fasteners such as hook and loop can be used as a fastening means instead of adhesive. In one embodiment of the bandage 100, hook fasteners could be bonded to the fastening tabs 110a to 110d on the interior side 106. Loop fabric can cover at least a portion of the exterior side 107. As long as the bandage 100 is sized for sufficient overlap as shown in FIG. 1G, such a mechanical fastening system could be employed. To the extent that aggravation of the skin might be a concern, other alternatives such as mushroom-type mechanical fasteners could be used

Mechanical fasteners could have the advantage at least for some applications of making a bandage more reusable. For example, a bandage could be constructed to withstand washing and steam heat by incorporating sturdy hook and loop type fasteners and sturdy layers and bonding methods. Such bandages can still fall within the scope of this invention

In other embodiments, other means of manufacture can be used. A bandage could be hand made or portions of it hand made. Other cutting means could be used. For example, flatbed dies or lasers can be used for cutting the bandages or its components. Such bandages can still fall within the scope of this invention

### Second Embodiment

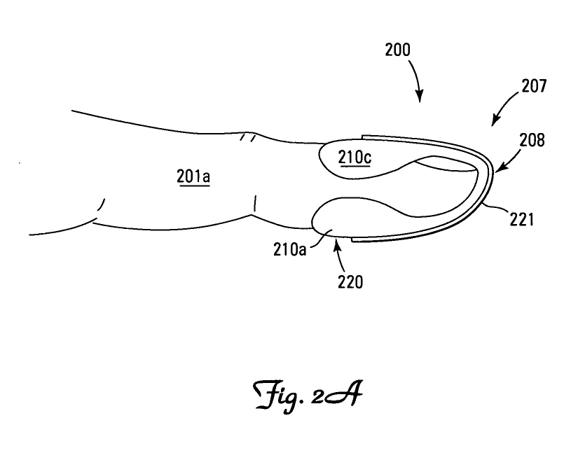

FIG. 2A shows a bandage 200 according to the second embodiment. The bandage 200 can substantially resemble the one shown in relation to FIGS. 1A to 1G, with at least one difference. The bandage 200 shown in FIG. 2A can have a splint 221 attached to the exterior side 207 of the exterior layer 220 of the bandage 200. The splint 221can be attached, for example, with adhesive.

As with the first embodiment of the bandage 100, the second embodiment of the bandage 200 can be applied to the index finger 201a. First the release liner (not shown) can be removed from the tabs (only tabs 210a and 210c shown) and the tabs located at one end of the bandage 200 (only tab 210a shown) can be applied to the finger 201a. At this stage of application, the bandage 200 can remain relatively straight in a longitudinal direction because of the splint 221. Next, the bandage 200 can be bent around the distal end 208 of the finger 201a. Finally, the tabs located at the other end of the bandage 200 (only tab 210c shown) can be affixed to the finger 201a to fully apply the bandage 200.

### Third Embodiment

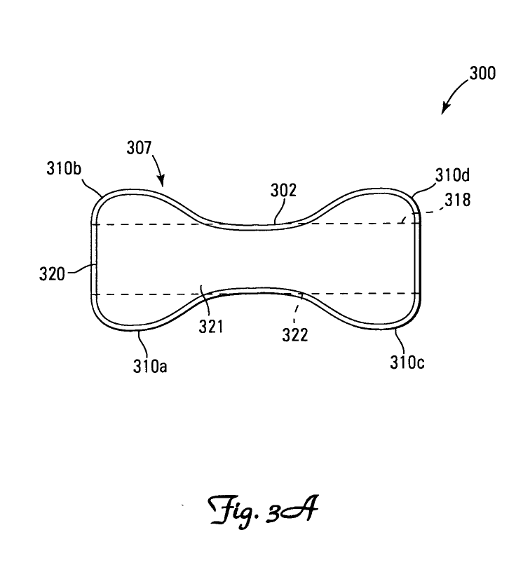

FIG. 3A shows the exterior side 307 of a bandage 300 according to the third embodiment. The bandage 300 can substantially resemble the one shown in relation to FIGS. 1A to 1G, with certain differences. The shape of the bandage 300 is defined by its peripheral edge 302 and defines an interior side (not shown) and an exterior side 307. Fastening tabs 310a, 310b, 310c and 310d can be formed as lateral extensions of the exterior layer 320 beyond the edge of the interior layer 318. The bandage 300 shown in FIG. 3A can have a splint 321 that is shaped by the peripheral edge 322 differently from the shape of the splint 121 shown in relation to FIGS. 1A to 1G. Moreover, the splint 321 can be mounted on the exterior layer 320 similar to the splint 221 shown in relation to FIG. 2A. The exterior layer 320 could be made of a clear or opaque material. Such a material could be a transparent perforated ethylene vinyl acetate tape coated on one side with an acrylate adhesive. Such a tape is offered by 3M® with a product number of 1527.

The shape of the splint 321 shown in FIG. 1A can offer certain advantages for certain applications. Such a shape could further reinforce the tabs 110a to Hod thereby further lessening the potential for the tabs 110a to 110d folding onto themselves or becoming attached unintentionally to certain surfaces

For such a bandage 300 it could be preferable to use a thinner material for the splint 321. A thinner material for the splint 321 could better provide conformity with the target surface. For most applications a thickness from approximately 0.001 to 0.010 might be most suitable. For some applications, however, such a splint can even be less than 0.001 inch thick.

Modifications

The present invention should not be considered limited to the particular examples described above, but rather should be understood to cover all aspects of the invention as fairly set out in the claims arising from this application. For example, while suitable sizes, materials, packaging and the like have been disclosed in the above discussion, it should be appreciated that these are provided by way of example and not of limitation as a number of other sizes, materials, fasteners, and so forth may be used without departing from the invention. Various modifications as well as numerous structures to which the present invention may be applicable will be readily apparent to those of skill in the art to which the present invention is directed upon review of the present specifications. The claims which arise from this application are intended to cover such modifications and structures.

We claim:

1. A bandage, comprising:
(a) a supple backing layer having first and second major surfaces, and
(b) a longitudinally elongated elastic splint adhesively secured to the first major surface of the backing.

2. The bandage of claim 1, further comprising a layer of padding secured onto a major surface of the backing layer.

3. The bandage of claim 1, further comprising a layer of a pressure sensitive adhesive on a major surface of the backing layer.

4. The bandage of claim 2, further comprising a layer of a pressure sensitive adhesive on a major surface of the backing layer.

5. The bandage of claim 3, further comprising a release liner over exposed areas of the layer of pressure sensitive adhesive.

6. The bandage of claim 4, further comprising a release liner over exposed areas of the layer of pressure sensitive adhesive.

7. The bandage of claim 1 wherein the backing layer has (i) a longitudinally elongated central area having a first longitudinal end and a second longitudinal end, and (ii) a pair of laterally extending mirror-image tabs proximate each longitudinal end of the central area.

8. The bandage of claim 2 wherein (a) the backing layer has (i) a longitudinally elongated central area having a first longitudinal end and a second longitudinal end, and (ii) a pair of laterally extending mirror-image tabs proximate each longitudinal end of the central area, and (b) the layer of padding overlays the central area so as to define at least two separate and distinct areas of exposed adhesive.

9. The bandage of claim 3 wherein the backing layer has (i) a longitudinally elongated central area having a first longitudinal end and a second longitudinal end, and (ii) a pair of laterally extending mirror-image tabs proximate each longitudinal end of the central area.

10. The bandage of claim 6 wherein (a) the backing layer has (i) a longitudinally elongated central area having a first longitudinal end and a second longitudinal end, and (ii) a pair of laterally extending mirror-image tabs proximate each longitudinal end of the central area, and (b) the layer of padding overlays the entire central area so as to define four separate and distinct areas of exposed adhesive.

11. The bandage of claim 3 wherein the pressure sensitive adhesive is a repositionable pressure sensitive adhesive.

12. The bandage of claim 3 wherein the pressure sensitive adhesive is other than a repositionable pressure sensitive adhesive.

13. The bandage of claim 2 wherein the padding is effective for absorbing wound exudate.

14. The bandage of claim 4 wherein the padding is effective for absorbing wound exudate.

15. The bandage of claim 6 wherein the padding is effective for absorbing wound exudate.

16. The bandage of claim 1 wherein the backing is cut from a continuous web.

17. The bandage of claim 1 wherein the splint is cut from a continuous web.

18. The bandage of claim 2 wherein the padding is cut from a continuous web.

19. The bandage of claim 2 wherein the backing, splint and padding are cut and attached to one another in a single press process.
